(19) United States
(12) Patent Application Publication
(10) Pub. No.: US 2019/0344049 A1
(43) Pub. Date: Nov. 14, 2019

(54) WRISTBAND WITH HEMOSTATIC VALVE FOR SHEATHLESS TRANSRADIAL PROCEDURE

(71) Applicant: BOSTON SCIENTIFIC SCIMED, INC., MAPLE GROVE, MN (US)

(72) Inventor: JAVIER PALOMAR-MORENO, GALWAY (IE)

(73) Assignee: BOSTON SCIENTIFIC SCIMED, INC., MAPLE GROVE, MN (US)

(21) Appl. No.: 16/405,964

(22) Filed: May 7, 2019

Related U.S. Application Data
(60) Provisional application No. 62/668,570, filed on May 8, 2018.

(51) Int. Cl. A61M 25/01; A61M 25/06; A61M 25/02

(52) U.S. Cl. CPC A61M 25/0113; A61M 25/09041; A61M 25/02; A61M 25/0662

(57) ABSTRACT

An assembly for use in sheathless transradial procedures includes a wristband that is adapted to be adjustably securable about a patient's wrist proximate an access point. A shaped member is secured relative to a midpoint of the wristband and is adapted to provide pressure proximate the access point. A hemostatic valve extends through the shaped member and is adapted to permit one or more elongate medical devices to extend therethrough.

### CROSS-REFERENCE TO RELATED APPLICATIONS

This application claims the benefit of priority under 35 U.S.C. § 119 of U.S. Provisional Application No. 62/668,570, filed May 8, 2018, the entire disclosure of which is hereby incorporated by reference.

### TECHNICAL FIELD

The disclosure is directed to devices for gaining access to the radial artery and more particularly is directed to devices for gaining access to the radial artery that enable sheathless transradial procedures.

### BACKGROUND

A variety of medical procedures are performed from within a patient's vascular system, with several different access points frequently used. In some cases, access to the vascular system is gained via the femoral artery in the leg. In some cases, access to the vascular system may be gained via the radial artery or other vasculature within the patient's arm such as but not limited to the ulnar artery. A variety of devices may be used for gaining access via the radial artery. Of the known medical devices and methods, each has certain advantages and disadvantages. There is an ongoing need to provide alternative medical devices as well as alternative methods for manufacturing and using medical devices.

### SUMMARY

The disclosure is directed to several alternative designs, materials and methods of manufacturing medical device structures and assemblies, and uses thereof. For example, the disclosure is directed to an assembly for use in sheathless transradial procedures. The Assembly includes a wristband that is adapted to be adjustably securable about a patient's wrist proximate an access point. A shaped member is secured relative to a midpoint of the wristband and is adapted to provide pressure proximate the access point. A hemostatic valve extends through the shaped member and is adapted to permit one or more elongate medical devices to extend therethrough.

Alternatively or additionally, the one or more elongate medical devices may include a guidewire.

Alternatively or additionally, the one or more elongate medical devices may include a guide catheter.

Alternatively or additionally, the wristband may include a first strap section extending in a first direction from the shaped member and a second strap section extending in a second, opposing, direction from the shaped member, and the first strap section is releasably and adjustably securable to the second strap section.

Alternatively or additionally, the first strap section may include one of hook fasteners and loop fasteners and the second strap section may include the other of hook fasteners and loop fasteners.

Alternatively or additionally, the shaped member may be resilient.

Alternatively or additionally, the shaped member may be inflatable.

Alternatively or additionally, the shaped member may be adapted to secure the hemostatic valve at an acute angle relative to the patient's forearm.

Alternatively or additionally, the hemostatic valve may be adapted to permit the assembly to slide distally and proximally relative to the one or more elongate medical devices extending therethrough.

Another example of the disclosure is an assembly for use in sheathless transradial procedures. The assembly includes a body portion that is adapted to provide pressure to an access point in order to reduce blood loss from the access point, a wristband that extends in either direction from the body portion and is adapted to be adjustably securable about a patient's wrist, and a hemostatic valve that extends through the body portion at an angle that is about the same as a corresponding angle of one or more elongate medical devices extending through the hemostatic valve and into the patient's forearm via the access point.

Alternatively or additionally, the access point may be formed using a hollow needle held at an angle relative to the patient's forearm, and the hemostatic valve extends through the body portion such that when the wristband is secured about the patient's wrist, the hemostatic valve extends at an angle relative to the patient's forearm that is about the same as the angle at which the hollow needle was held when forming the access point.

Alternatively or additionally, the body portion may include a resilient polymer.

Alternatively or additionally, the one or more elongate medical devices may include a guidewire and/or a guide catheter.

Alternatively or additionally, the wristband may include a first strap section and a second strap section, and the first strap section is releasably and adjustably securable to the second strap section.

Alternatively or additionally, the first strap section may include one of hook fasteners and loop fasteners and the second strap section may include the other of hook fasteners and loop fasteners.

Alternatively or additionally, the body portion may have a conical or frustoconical shape.

Alternatively or additionally, the body portion may have a pyramidal shape.

Another example of the disclosure is an assembly for use in sheathless transarterial procedures. The assembly includes a strap that is adapted to be adjustably securable about a patient's limb proximate an access point, a shaped member that is secured relative to a midpoint of the strap and that is adapted to provide pressure proximate the access point and a hemostatic valve that extends through the shaped member and is adapted to permit one or more elongate medical devices to extend therethrough.

Alternatively or additionally, the strap may be adapted to be adjustably securable about a patient's wrist or forearm.

Alternatively or additionally, the strap may be adapted to be adjustable securable about a patient's leg.

The above summary of some embodiments is not intended to describe each disclosed embodiment or every implementation of the present disclosure. The FIGS., and Description, which follow, more particularly exemplify these embodiments.

### BRIEF DESCRIPTION OF THE FIGURES

The disclosure may be more completely understood in consideration of the following description of in connection with the accompanying drawings, in which:

FIG. 1 is a perspective view of an assembly for use in sheathless transradial procedures in accordance with the disclosure;

FIG. 2 is a perspective view of the assembly for use in sheathless transradial procedures of FIG. 1, shown in use in accordance with the disclosure;

FIG. 3 is a perspective view of the assembly for use in sheathless transradial procedures of FIG. 1, shown in use in accordance with the disclosure;

FIG. 4 is a perspective view of the assembly for use in sheathless transradial procedures of FIG. 1, shown in use in accordance with the disclosure;

FIG. 5 is a schematic view of a portion of the assembly for use in sheathless transradial procedures in accordance with the disclosure;

FIG. 6 is a schematic view of a portion of the assembly for use in sheathless transradial procedures in accordance with the disclosure; and

FIG. 7 is a schematic view of a portion of the assembly for use in sheathless transradial procedures in accordance with the disclosure.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

Definitions of certain terms are provided below and shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a,” “an,” and “the” include or otherwise refer to singular as well as plural referents, unless the content clearly dictates otherwise. As used in this specification and the appended claims, the term “or” is generally employed to include “and/or,” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

A variety of medical procedures, including a variety of cardiac related procedures, are done via arterial access. In some cases, transradial coronary interventions may be viewed as safer than transfemoral coronary interventions due at least in part to reduced bleeding complications. In some instances, a sheathless procedure, in which no introducer sheath is used, and a guide catheter is inserted directly into the radial artery, may be used in patients having small radial arteries. For example, radial arteries may vary, between patients, from a diameter of about 1.5 millimeters (mm) to about 4 mm. As transradial coronary interventions are generally performed with 6 Fr catheters having an outer diameter of 2 mm and a corresponding sheath outer diameter of 2.7 mm, it will be appreciated that in some cases, a sheathless procedure may be a better choice for some patients.

Procedures for sheathless transradial access have been developed. In some cases, for example, cardiologists utilize a partially inflated balloon within the guide catheter, with the distal end of the balloon partially protruding from the distal end of the guide catheter as a sort of atraumatic tip. This is known as BAT, or balloon-assisted tracking. In some cases, the balloon also helps to smoothly guide the guide catheter through the vasculature, rather than potentially causing trauma against the arterial walls. In some cases, the use of dilators with the guide catheter can eliminated a need for an introducer sheath. In sheathless transradial procedures such as these, in some cases blood loss may occur at the access point, and the cardiologist may apply pressure to the access point with their finger in order to reduce blood loss.

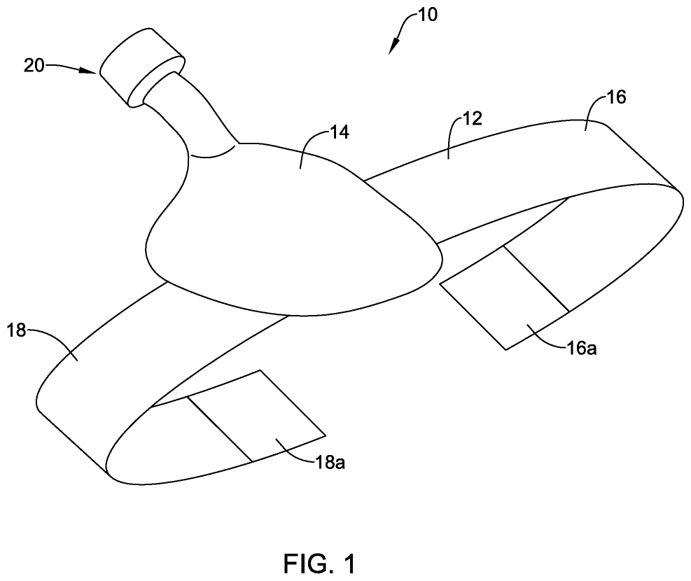

FIG. 1 is a perspective view of an assembly 10 that is adapted for use in sheathless transradial procedures. The assembly 10 includes a wristband 12 that is adapted to be adjustable securable about a patient's wrist, proximate the access point. In some cases, the assembly 10 is intended to overlie the access point. The assembly 10 includes a shaped member, or body portion 14 that is adapted to provide pressure proximate the access point. In some cases, the shaped member 14 may be resilient, and may be formed of a resilient polymer. In some instances, the shaped member 14 may be formed of Nylon (polyamide) or PEBA (polyether block polyamide) in which the relative flexibility and strength (hardness) may be adjusted by controlling the relative amounts of polyamide. Other suitable materials include but are not limited to FEP (fluorinated ethylene propylene), PET (polyethylene terephthalate), PTFE (polytetrafluoroethylene), PEEK (polyetheretherketone), PU (polyurethane), EVA (ethylene vinyl acetate), PEI (polyetherimide), polyethylene, silicones and the like. In some instances, the shaped member 14 may be inflatable, or include an inflatable portion that may be inflated and/or deflated in order to control the relative pressure being applied to an access point.

In some cases, for example, the wristband 12 includes a first strap section 16 that extends in a first direction from the shaped member or body portion 14, and a second strap section 18 that extends in a second, opposing direction from the shaped member or body portion 14. In some instances, the first strap section 16 is releasably and adjustable securable to the second strap section 18 in order to fasten the assembly 10 in place relative to a patient's wrist. The wristband 12 may be formed of any suitable woven or webbed material, for example, and may be any of a variety of different polymers. In some cases, the wristband 12 may be formed of polyurethane thermoplastic elastomers. Nylon, polyethylene and other materials may also be used.

In some cases, the first strap section 16 may include a region 16a that includes one of hook fasteners and loop fasteners while the second strap section 18 may include a region 18a that includes the other of hook fasteners and loop fasteners. The region 16a and the region 18a may, in combination, functionally provide hook and loop fasteners such as but not limited to those available commercially as Velcro®. While not expressly illustrated, in some cases, both an inner surface and an outer surface of the region 16a and an inner both an inner surface and an outer surface of the region 18a may include hook and loop fasteners such that the first strap section 16 and the second strap section 18 may be releasably and adjustably secured together regardless of which section overlies the other. In some cases, one strap section 16, 18 may have holes punched in it while the other strap section 16, 18 includes a buckle. A single point quick release buckle, such as those used in automobiles, may be used. In some cases, a clip-in system, such as that used in baby strollers and buggies, in which a strap can be pulled to tighten, may also be used.

The assembly 10 includes a hemostatic valve 20 that extends through the shaped member 14 and is adapted to permit one or more elongate medical devices to extend therethrough. For example, the one or more elongate medical devices may include but not be limited to a guidewire or a guide catheter. Other interventional devices are also contemplated. In some cases, the shaped member 14 is adapted to secure the hemostatic valve 20 at an acute angle relative to the patient's forearm. In some instances, the hemostatic valve 20 may be adapted to permit the assembly 10 to slide distally and proximally relative to the one or more elongate medical devices extending therethrough.

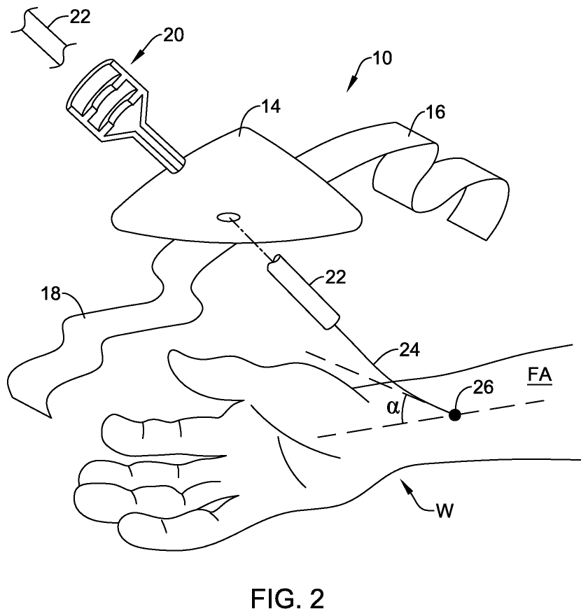

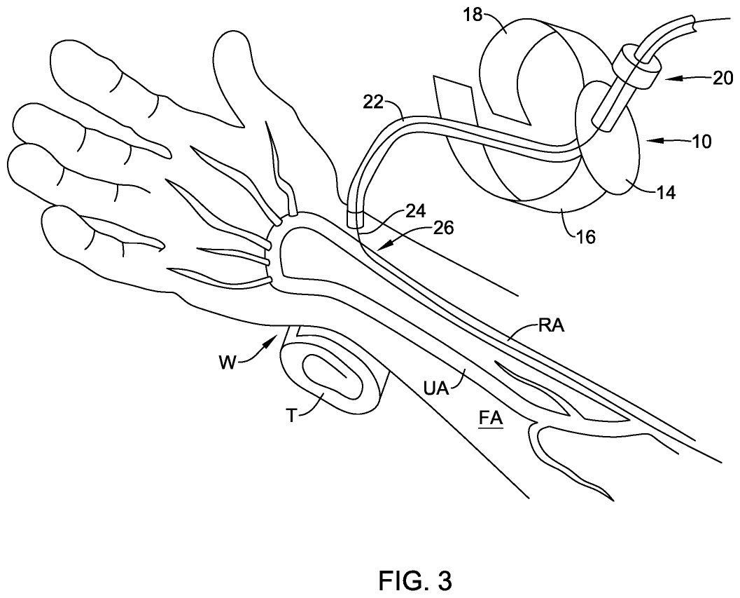

In use, a nurse or other medical professional may load the assembly 10 onto a guide catheter by passing the guide catheter through the hemostatic valve. The assembly 10 may be slid proximally in order to move the assembly 10 out of the way. The assembly 10 may subsequently be slid down the guide catheter into position against the patient's wrist, and can be secured in place via an interaction between the first strap section 16 and the second strap section 18. In some cases, a rolled up towel T may be placed under the wrist W for support. FIG. 2 shows the assembly 10 in place on a guide catheter 22. The guide catheter 22 is itself disposed on a guidewire 24 that enters an access point 26 that in some cases is just proximal of the patient's wrist W. In some cases, the access point 26 may have been formed using a hollow needle that was held at an angle that approximates the angle α shown in FIG. 2 between the guidewire 24 and the patient's forearm FA. FIG. 3 is similar to FIG. 2, but shows the radial artery RA and the ulnar artery UA within the patient's forearm FA.

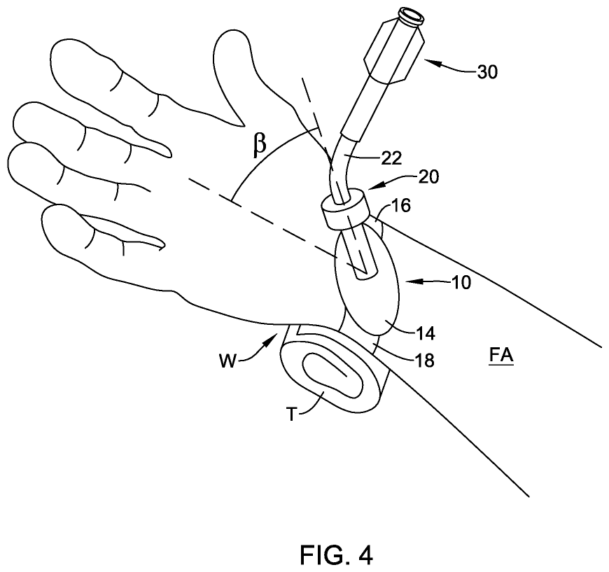

FIG. 4 shows the assembly 10 in position relative to the patient's forearm FA. The first strap section 16 and the second strap section 18 are secured together about the patient's wrist W. As can be seen, the guide catheter 22 includes a proximal hub 30. Prior to sliding the assembly 10 down the guide catheter 22 into position against the patient's wrist W, the assembly 10 may be as far proximal as to contact the proximal hub 30. The assembly 10 may be slid into position, as shown in FIG. 4, once a distal end of the guide catheter 22 has been positioned in the ostium of the coronary artery to be treated, for example. The shaped member 14 is able to provide a constant, but light, pressure relative to the access point 26 in order to minimize blood loss. In some cases, as illustrated, a rolled up towel T may be placed under the patient's wrist W to better support the patient's wrist W while allowing the hand to roll under.

It can be seen in FIG. 4 that the shaped member 14 holds the hemostatic valve 20 at an angle β between the hemostatic valve 20 and the patient's forearm FA. In some cases, and as may be appreciated by comparing FIG. 2 and FIG. 4, that the angle β between the hemostatic valve 20 and the patient's forearm FA (shown in FIG. 4) is the same or about the same as angle α (shown in FIG. 2) between the guidewire 24 and the patient's forearm FA. In some cases, this helps to reduce possible trauma to the patient.

The assembly 10 has been described with respect to sheathless transradial procedures. In some cases, it is contemplated that the assembly 10 may more generally used in procedures gaining access to an artery or vein within a patient's limb. In some cases, the assembly 10 may be used on a patient's arm when gaining access to an artery or a vein within the arm. For example, the assembly 10 may be used when accessing a radial artery or an ulnar artery within the patient's forearm, or perhaps the brachial artery in the patient's upper arm. In some cases, the assembly 10 may be used on a patient's leg when gaining access to an artery or a vein within the leg, such as but not limited to, the femoral artery in the upper leg.

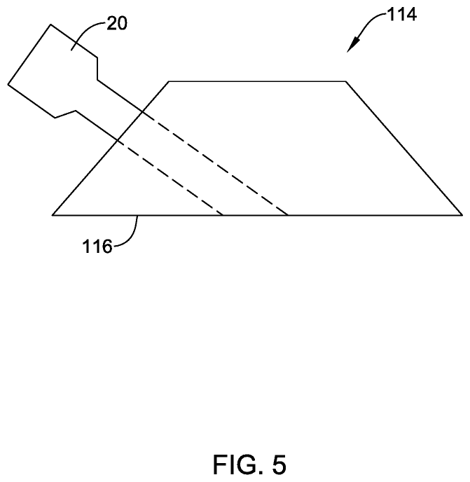

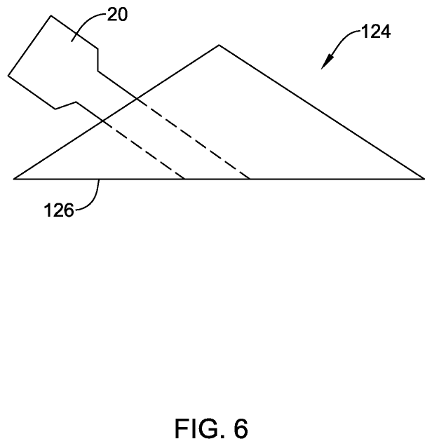

FIGS. 5 through 7 provide additional examples of particular configurations for the shaped member 14. Each of the shaped members 114, 124, 134 shown in FIGS. 5-7 may be considered as options for the shaped member 14 shown in FIGS. 1-4, and may be used in combination with the wristband 12 and the hemostatic valve 20 also shown in FIGS. 1-4. FIG. 5 shows a shaped member 114 that has a frustroconical shape, which generally is a conical shape with the tip of the cone removed. The shaped member 114 has a lower surface 116 that is adapted to provide pressure against the access point 26. While shown as flat, in some cases the lower surface 116 may instead have a curved profile to better apply pressure to the access point 26 while providing less pressure to skin near but not at the access point 26. FIG. 6 shows a shaped member 124 that has more of pyramidal shape. The shaped member 124 has a lower surface 126 that is adapted to provide pressure against the access point 26. While shown as flat, in some cases the lower surface 126 may instead have a curved profile to better apply pressure to the access point 26 while providing less pressure to skin near but not at the access point 26.

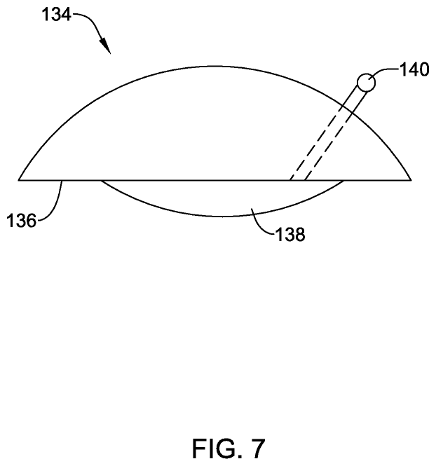

FIG. 7 shows a shaped member 134 that has a generally rounded shape, but with a flat lower surface 136. In some cases, as illustrated, the lower surface 136 may include an inflatable portion 138 that may be inflated in order to provide additional pressure to the access point 26 or deflated in order to provide less pressure to the access point 26. The shaped member 134 includes a pneumatic port 140 that may be used to provide inflation fluid to the inflatable portion 138, or to remove inflation fluid from the inflatable portion 138. In some cases, the inflation fluid may be a liquid such as saline. In some instances, the inflation fluid may simply be pressurized air, or a relatively inert gas such as nitrogen.

It will be appreciated that a variety of different materials may be used in forming the devices described herein. In some embodiments, for example, the devices may include any suitable polymeric material, including biocompatible materials such as polyurethane or silicone. Other suitable polymers include but are not limited to polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

The devices described herein may be formed of any suitable desired material, such as a biocompatible material including biostable, bioabsorbable, biodegradable or bioerodible materials, including in some cases one or more metals. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Those skilled in the art will recognize that the present disclosure may be manifested in a variety of forms other than the specific embodiments described and contemplated herein. Accordingly, departure in form and detail may be made without departing from the scope and spirit of the present disclosure as described in the appended claims.

What is claimed is:

1. An assembly for use in a sheathless transradial procedure, the assembly comprising:
a wristband adapted to be adjustably securable about a patient's wrist proximate an access point;
a shaped member secured relative to a midpoint of the wristband, the shaped member adapted to provide pressure proximate the access point; and
a hemostatic valve extending through the shaped member, the hemostatic valve adapted to permit one or more elongate medical devices to extend therethrough.

2. The assembly of claim 1, wherein the one or more elongate medical devices comprises a guidewire.

3. The assembly of claim 1, wherein the one or more elongate medical devices comprises a guide catheter.

4. The assembly of claim 1, wherein the wristband includes a first strap section extending in a first direction from the shaped member and a second strap section extending in a second, opposing, direction from the shaped member, and the first strap section is releasably and adjustably securable to the second strap section.

5. The assembly of claim 4, wherein the first strap section includes one of hook fasteners and loop fasteners and the second strap section includes the other of hook fasteners and loop fasteners.

6. The assembly of claim 1, wherein the shaped member is resilient.

7. The assembly of claim 1, wherein the shaped member is inflatable.

8. The assembly of claim 1, wherein the shaped member is adapted to secure the hemostatic valve at an acute angle relative to the patient's forearm.

9. The assembly of claim 1, wherein the hemostatic valve is adapted to permit the assembly to slide distally and proximally relative to the one or more elongate medical devices extending therethrough.

10. An assembly for use in a sheathless transradial procedure, the assembly comprising:
a body portion adapted to provide pressure to an access point in order to reduce blood loss from the access point;
a wristband extending in either direction from the body portion, the wristband adapted to be adjustably securable about a patient's wrist; and
a hemostatic valve extending through the body portion at an angle that is about the same as a corresponding angle of one or more elongate medical devices extending through the hemostatic valve and into the patient's forearm via the access point.

11. The assembly of claim 10, wherein the access point is formed using a hollow needle held at an angle relative to the patient's forearm, and the hemostatic valve extends through the body portion such that when the wristband is secured about the patient's wrist, the hemostatic valve extends at an angle relative to the patient's forearm that is about the same as the angle at which the hollow needle was held when forming the access point.

12. The assembly of claim 10, wherein the body portion comprises a resilient polymer.

13. The assembly of claim 10, wherein the one or more elongate medical devices comprises a guidewire and/or a guide catheter.

14. The assembly of claim 10, wherein the wristband includes a first strap section and a second strap section, and the first strap section is releasably and adjustably securable to the second strap section.

15. The assembly of claim 14, wherein the first strap section includes one of hook fasteners and loop fasteners and the second strap section includes the other of hook fasteners and loop fasteners.

16. The assembly of claim 10, wherein the body portion has a conical or frustoconical shape.

17. The assembly of claim 10, wherein the body portion has a pyramidal shape.

18. An assembly for use in a sheathless transarterial procedure, the assembly comprising:
a strap adapted to be adjustably securable about a patient's limb proximate an access point;
a shaped member secured relative to a midpoint of the strap, the shaped member adapted to provide pressure proximate the access point; and
a hemostatic valve extending through the shaped member, the hemostatic valve adapted to permit one or more elongate medical devices to extend therethrough.

19. The assembly of claim 18, wherein the strap is adapted to be adjustably securable about a patient's wrist or forearm.

20. The assembly of claim 18, wherein the strap is adapted to be adjustable securable about a patient's leg.
